(19) United States
(12) Patent Application Publication
(10) Pub. No.: US 2021/0369897 A1
(43) Pub. Date: Dec. 2, 2021

(54) FOOTWEAR SANITIZATION SYSTEM

(71) Applicant: Justrite Manufacturing Company, L.L.C. dba Justrite Safety Group, Deerfield, IL (US)

(72) Inventors: Brian B. Evans, Homewood, IL (US); Nancy Huber, Tomahawk, WI (US)

(21) Appl. No.: 17/333,757

(22) Filed: May 28, 2021

Related U.S. Application Data
(60) Provisional application No. 63/031,548, filed on May 28, 2020.

(51) Int. Cl. A61L 2/18

(52) U.S. Cl. CPC A61L 2/18; A61L 2202/17

(57) ABSTRACT

A footwear sanitization system or floormat which may have one or more zones to. The zones may include a disinfecting zone, a drying zone, a scraping zone that are used to perform different procedures on the footwear for the purposes of cleaning or sanitizing the footwear when the footwear is in or on the zone.

### CROSS REFERENCE TO RELATED APPLICATION

This application claims the benefit of U.S. Provisional Application No. 63/031,548 filed May 28, 2020, the contents of which are hereby incorporated by reference in its entirety.

### BACKGROUND

Many diseases, such as viruses and Influenza, can spread through the air. For example, disease may spread when infected patients cough, sneeze, talk, or the like, resulting in spewing nasal and/or throat secretions into the air. Furthermore, some diseases may then travel through an environment and further land on various surfaces. For example, in a hospital setting diseases may land on computer mice, trash cans, sickbed rails, doorknobs, or the like.

General practices around the world provide guidelines to hand washing and social distancing to prevent the spread of contagious diseases, such as viruses, Influenza, or the like. Furthermore, high touch surfaces, such as door handles, sickbed handrails, countertops, etc. can be sanitized in order to limit the spread of disease. However, more sanitization systems may be necessary to further limit the spread of disease. Some such systems are described in this invention.

### SUMMARY

In an example of the disclosure, a footwear sanitization floormat is disclosed. The floormat includes a disinfecting zone and a drying zone. The disinfecting zone includes a tray with raised walls for holding a disinfecting fluid, and the disinfecting zone is configured to immerse a portion of footwear placed upon the disinfecting zone in the disinfecting fluid. The drying zone includes one or more materials configured to absorb fluid from the footwear placed on the drying zone. The disinfecting zone and the drying zone are configured to secure together to form a floormat having multiple zones.

In another example of the disclosure, a sanitizing floormat is disclosed. The floormat includes a disinfecting zone and a drying zone. The disinfecting zone includes a tray with raised walls for holding a disinfecting fluid. The disinfecting zone is also configured to immerse a portion of footwear placed in the disinfecting fluid. The drying zone includes one or more materials configured to absorb fluid, including the disinfecting fluid, from footwear placed upon the drying zone.

### BRIEF DESCRIPTION OF THE DRAWINGS

The following drawings are illustrative of particular embodiments of the invention and therefore do not limit the scope of the invention. The drawings are not necessarily to scale (unless so state) and are intended for use with the explanations in the following detailed description. Embodiments of the invention will hereinafter be described in conjunction with the appended drawings, wherein like numerals denote like elements.

FIG. 1 illustrates an exemplary embodiment of a show wet dip disinfection system.

FIG. 2 illustrates a side view of the show wet dip disinfection system of FIG. 1.

### DESCRIPTION

The following detailed description is exemplary in nature and is not intended to limit the scope, applicability, or configuration of the invention in any way. Rather, the following description provides some practical illustrations form implanting various embodiments of the present invention. Those skilled in the art will recognize that many of the noted examples have a variety of suitable alternatives. Embodiments described herein generally relate to systems and methods relating to a shoe wet dip disinfection system configured to disinfect shoes, provide foot hygiene, or the like while complying with CDC and BOMA guidelines.

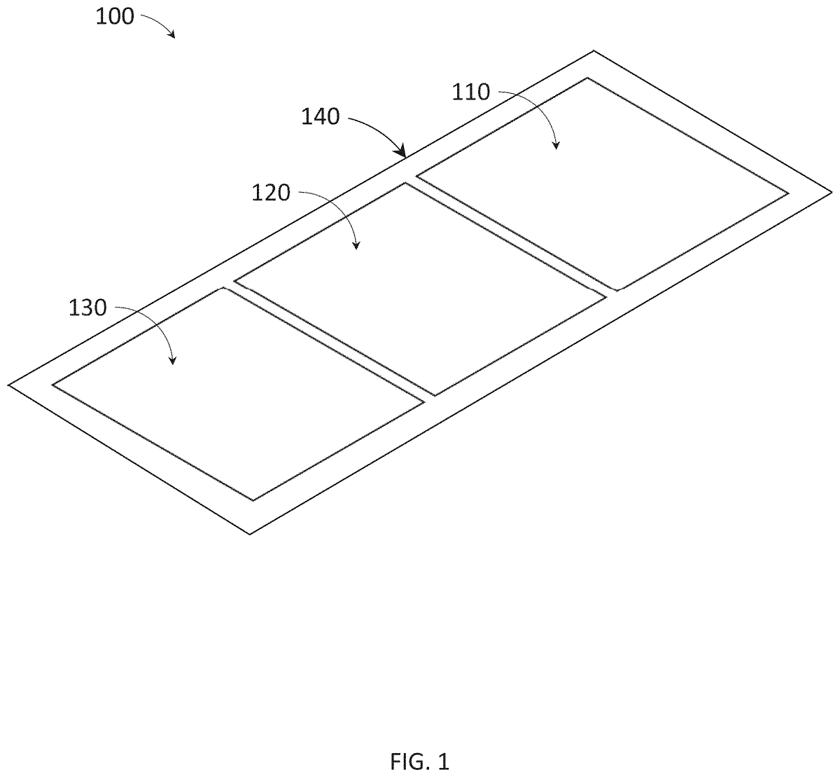

FIG. 1 provides an exemplary embodiment of a shoe wet dip disinfection system 100 (SWDD system 100) for disinfecting shoes, providing foot hygiene, or the like. SWDD system 100 may comprise one or more zones, such as Cleaning Zone 110, Disinfecting Zone 120, and Drying Zone 130, as shown in FIG. 1. In some embodiments, people or the like may traverse the zones from Cleaning Zone 110 to Drying Zone 130 to receive safely receive disinfection on their footwear. As shown in FIG. 1, SWDD system 100 may comprise a housing 140 for the zones. Alternatively, one or more zones may be separate from one another, such as described herein.

In some embodiments, indicia may be provided on and/or near one or more zones (e.g. Cleaning Zone 110, Disinfecting Zone 120, and Drying Zone 130) to provide information to a user on what process the zone is performing, what to do during each process or the like. Indicia may comprise letters, symbols, pictographs, or the like. For example, indicia indicating “Please Wipe Footwear Here” or the like may be placed adjacent to Cleaning Zone 110; indicia indicating “Please Wash/Sanitize Footwear Here” may be placed adjacent to Disinfecting Zone 120; and indicia indicating “Please Dry Footwear Here” may be placed adjacent to Drying Zone 130.

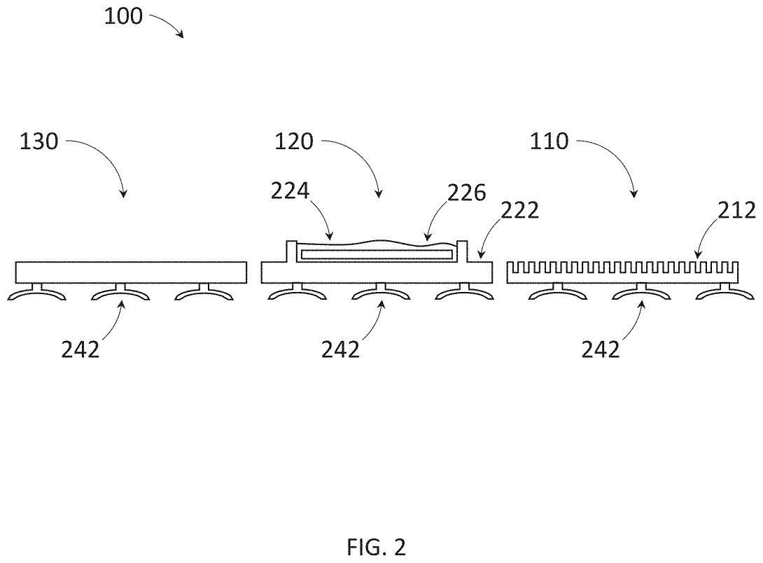

FIG. 2 provides an exemplary side view of SWDD system 100. As shown, Cleaning Zone 110 may comprise a scraping mechanism 212, such as stiff rubber bristles or the like. Cleaning Zone 110 may be configured to remove debris from footwear or the like, such as dirt, soil, debris found on the bottom of footwear, etc. In some embodiments, portions of Cleaning Zone 110 may be removable and/or replaceable, such as scraping mechanism 212. For example, if scraping mechanism 212 becomes saturated with debris or has a reduction in functionality (e.g. over time from use) it may be beneficial to remove scraping mechanism 212 to replace it with a new scraping mechanism, clean/fix scraping mechanism 212, or the like.

Disinfecting Zone 120 may be configured to disinfect footwear or the like, such as to remove, deactivate or kill various pathogens (e.g. COVID-19) which may be located on the footwear. Disinfecting Zone 120 may comprise a tray 222 beveled on all 4 sides with raised walls to accommodate disinfectant 224. In some embodiments, disinfectant 224 may comprise a quaternary ammonia disinfectant and/or other chemicals known to provide disinfection. Various disinfectants may be used depending on the environment that SWDD system 100 is located in as well as one or more diseases of interest. In such embodiments, disinfectant 224 may be provided in the tray 222 up to a predetermined level, such as a predetermined level to allow for the bottom of footwear or the like to be coated with disinfectant. The predetermine level may allow for enough disinfectant 224 to be present such that the soles of footwear or the like come in contact with the disinfectant. Additionally or alternatively, the predetermined level may allow for footwear or the like to not excessively slosh disinfectant 224 or receive disinfectant 224 in undesired locations, such as inside the footwear. Furthermore, the tray may comprise markings and/or indicia to provide information on how much disinfectant is needed to achieve the predetermined level.

Disinfecting Zone 120 may additionally comprise an insert 226, such as insert 226, for anti-sloshing of disinfectant 224, preventing spillage of disinfectant 224, and/or preventing evaporation of disinfectant 224. In some embodiments, insert 226 may be removable and/or replaceable. For example, if insert 226 becomes saturated with debris or has a reduction in functionality (e.g. overtime from use) it may be beneficial to remove insert 226 to replace it with a new insert, clean insert 122, or the like.

Drying Zone 130 may be configured to remove any excess disinfectant on footwear or the like after being disinfected in Disinfecting Zone 120. Drying Zone 130 may comprise one or more materials which can absorb excess liquid (e.g. disinfectant 224) off of footwear. Drying Zone 130 may provide a safe transition for a user off of SWDD system 100 such as to prevent slipping when traversing off of SWDD system 100. Additionally or alternatively, Drying Zone 130 may be configured to remove any excess chemicals from Disinfecting Zone 120 to limit tracking such chemicals off of SWDD systems 100. In some embodiments, Drying Zone 130 may be removable and/or replaceable. For example, if Drying Zone 130 becomes saturated with debris, disinfectant, or has a reduction in functionality (e.g. overtime from use) it may be beneficial to remove Drying Zone 130 to replace it with a new Drying Zone, clean Drying Zone 130, or the like.

Various SWDD systems 100 may be located on a floor, such as in a high foot traffic area (e.g. entrances and exits of rooms or buildings). In such embodiments, various portions of the SWDD system may be fixed to the floor in order to reduce movement of such various portions of the SWDD system when being used. In some embodiments, the SWDD system may be permanently built into or fixed to the floor. Alternatively, the SWDD system may be adjustable or movable.

In some embodiments, the SWDD system 100 may be fixed to the floor using various adhesives Additionally or alternatively, the SSWD system 100 may be may comprise one or more adherence devices 242 to help reduce movement when being used. The one or more adherence devices 242 may comprise suction cups, a textured surface underneath one or more portions of SWDD system 100 (e.g. one or more zones), or the like.

In some embodiments, all three zones (e.g. Cleaning Zone 110, Disinfecting Zone 120, and Drying Zone 130) may not be present. For example, in environments wherein debris (e.g. mud, dirt, etc.) is not perceived to be an issue, SWDD system 100 may comprise Disinfecting Zone 120, Drying Zone 130, and not Cleaning Zone 110. Furthermore, SWDD system 100, as discussed herein, can be used in a variety of situations. For example, a SWDD system may be used in areas with high foot traffic, such as train stations, hospitals, commercial shopping centers, schools, hotels, shops, offices, homes, or the like. SWDD systems may be used during or after events which increase the risk of disease, such as concerts, sporting events, or the like.

Various embodiments have been described. Such examples are non-limiting, and do not define or limit the scope of the invention.

What is claimed:

1. A footwear sanitization floormat, comprising:
a disinfecting zone comprising a tray with raised walls for holding a disinfecting fluid, the disinfecting zone configured to immerse a portion of footwear placed upon the disinfecting zone in the disinfecting fluid;
a drying zone comprising one or more materials configured to absorb fluid from footwear placed on the drying zone.

2. The footwear sanitization floormat of claim 1, wherein the disinfecting zone and the drying zone are secured within a housing to form a single floormat having multiple zones.

3. The footwear sanitization floormat of claim 2, further comprising a cleaning zone including a scraping mechanism configured to remove debris from footwear placed upon the cleaning zone, the cleaning zone being located proximate the disinfecting zone.

4. The footwear sanitization floormat of claim 3, wherein the disinfecting zone is detachable from the housing containing the drying zone and the cleaning zone.

5. The footwear sanitization floormat of claim 3, wherein the scraping mechanism is removable from the cleaning zone.

6. The footwear sanitization floormat of claim 1, wherein the disinfecting zone further includes an insert configured to prevent the disinfecting fluid from sloshing within the tray.

7. The footwear sanitization floormat of claim 6, wherein the insert is removable from the disinfecting zone.

8. The footwear sanitization floormat of claim 1, wherein the footwear sanitization floormat is affixed to a floor using one or more adhesive devices.

9. The footwear sanitization floormat of claim 8, wherein the one or more adhesive devices comprise suction cups.

10. The footwear sanitization floormat of claim 9, wherein the one or more adhesive devices comprise a textured surface.

11. The footwear sanitization floormat of claim 1, wherein the raised walls are beveled.

12. The footwear sanitization floormat of claim 1, wherein the footwear sanitization floormat comprises vinyl.

13. The footwear sanitization floormat of claim 1, wherein the disinfecting zone and the drying zone have substantially the same width, the drying zone extending for a length greater than the disinfecting zone.

14. The footwear sanitization floormat of claim 1, wherein the tray is configured to hold a predetermined level of disinfecting fluid and wherein the tray comprises markings to indicate the predetermined level of disinfecting fluid.

15. The footwear sanitization floormat of claim 1, further comprising markings applied to at least one of the disinfecting zone or the drying zone, the markings indicating instructions for a user of the footwear sanitization floormat.

16. The footwear sanitization floormat of claim 1, wherein the tray of the disinfecting zone further comprises flexible bristles configured to flex under pressure from footwear and permit the immersion of the portion of the footwear placed upon the disinfecting zone.

17. A sanitizing floormat comprising:
a disinfecting zone comprising a tray with raised walls for holding a disinfecting fluid, the disinfecting zone configured to immerse a portion of footwear placed upon the disinfecting zone in the disinfecting fluid;
a drying zone comprising one or more materials configured to absorb fluid, including the disinfecting fluid, from footwear placed upon the drying zone.

18. The sanitizing floormat of claim 17, further comprising a cleaning zone including a scraping mechanism configured to remove debris from footwear placed upon the cleaning zone, the cleaning zone being located proximate the disinfecting zone.

19. The sanitizing floormat of claim 18, wherein the disinfecting zone is detachable from a housing comprising the drying zone and the cleaning zone.

20. The sanitizing floormat of claim 18, wherein the scraping mechanism is removable from the cleaning zone.

21. The sanitizing floormat of claim 17, wherein the disinfecting zone further includes an insert configured to prevent the disinfecting fluid from sloshing within the tray.

22. The sanitizing floormat of claim 21, wherein the insert is removable from the disinfecting zone.
